(19) United States
(12) Patent Application Publication
(10) Pub. No.: US 2017/0333682 A1
(43) Pub. Date: Nov. 23, 2017

(54) Tearaway Sheath Introducer and Method

(71) Applicant: VascuTech Medical LLC, Oaks, PA (US)

(72) Inventor: Mahase Nardeo, Oaks, PA (US)

(73) Assignee: VascuTech Medical LLC, Oaks, PA (US)

(21) Appl. No.: 15/598,560

(22) Filed: May 18, 2017

Related U.S. Application Data
(60) Provisional application No. 62/340,231, filed on May 23, 2016.

(51) Int. Cl. A61M 25/06; A61B 17/34; A61M 25/00; A61M 29/02

(52) U.S. Cl. CPC A61M 25/0668; A61M 29/02; A61B 17/3498; A61M 25/0097

(57) ABSTRACT

Disclosed is a tearaway sheath introducer. The sheath includes a sheath hub disposed on an end thereof, wherein the sheath is configured to receive a dilator therethrough, further wherein the dilator comprises a hub with a locking mechanism. The sheath hub is configured to retain a cap set having a molded valve therein. The cap set and the valve can be ultrasonically welded to the sheath hub such that the cap set and the valve are integral to the sheath hub. The valve prevents blood from coming out of a patient's incision site after the dilator is removed from the sheath, but before a catheter is inserted. In this way, the present invention improves the process of introducing catheters into the blood vessels.

### CROSS-REFERENCE TO RELATED APPLICATIONS

This application claims the benefit of U.S. Provisional Patent Application No. 62/340,231, filed May 23, 2016, which is hereby incorporated by reference in its entirety.

### FIELD OF THE INVENTION

The present invention relates generally to medical devices, namely, catheters. More particularly, the present invention is directed to a tearaway sheath introducer having a valve that is molded onto a cap.

### BACKGROUND OF THE INVENTION

Tearaway sheath is generally used to introduce soft catheters (e.g., dialysis catheters) into the blood vessels. More specifically, an introducer sheath with a dilator device is passed over the guide wire to enlarge the venotomy or an incision site on a patient. Once the venotomy is enlarged, the dilator is removed, and then the catheter is inserted into the sheath. Thereafter, the sheath is removed by being split apart. The existing method, however, is disadvantageous in that a patient's blood could spurt from the vessel when the dilator is removed and before the catheter is inserted into the sheath. Additionally, air or other outside elements could be aspirated into the vessel, which is not desirable. In this regard, the invention described herein addresses these problems.

### SUMMARY OF THE INVENTION

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as a prelude to the more detailed description that is disclosed later.

In one embodiment, the present invention comprises a sheath with a sheath hub disposed on an end thereof, wherein the sheath is configured to receive a dilator therethrough, further wherein the dilator comprises a hub with a locking mechanism. The sheath hub comprises a cap set having a molded valve thereon. In some embodiments, the cap set and the valve are ultrasonically welded to the sheath hub. The sheath with the dilator is passed over a guide wire at an incision site of a patient. The valve is composed of a resilient material so that it is configured to allow the dilator to be pulled out therefrom while preventing blood from escaping after the dilator is removed, but before the catheter is inserted. In this way, the present invention improves the process of introducing catheters into the blood vessels by preventing blood from coming out and outside elements from being introduced into the sheath.

### BRIEF DESCRIPTION OF THE DRAWINGS

FIG. 1 depicts a tipped sheath that is molded with a hub to house a valve.

FIG. 2 shows a view of two cap sections assembled together to form a complete cap set.

FIG. 3 shows a view of a valve of the present invention.

FIG. 4 shows a rear view of the valve after it is molded onto the cap set.

FIG. 5 shows a front view of the valve after it is molded onto the cap set.

FIG. 6 shows a view of a dilator with a hub.

FIG. 7 shows a view of the complete assembly of the tearaway sheath introducer.

### DETAILED DESCRIPTION OF THE INVENTION

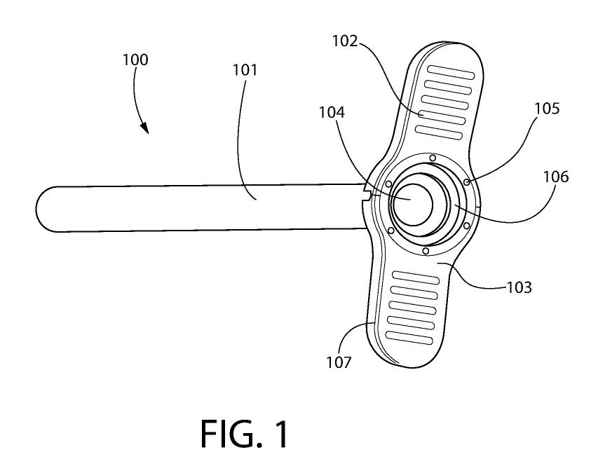

Referring now to FIG. 1, there is shown an exemplary embodiment of the tipped sheath 100 of the present invention. In a preferred embodiment, the sheath 100 is composed of high-density polyethylene (HDPE) or another suitable material. The sheath 100 comprises a substantially open tubular structure 101, defining an interior volume, wherein the tubular structure 101 comprises a circular cross section having a uniform diameter over its length. The tubular structure 101 comprises a first end and a second end, wherein the second end comprises a hub 103 molded thereon. In some embodiments, the hub 103 comprises a base member that connect to the tubular structure 101, wherein the base member comprises a substantially cylindrical shape. The tubular structure 101 is centrally located on the hub 103, wherein the hub 103 comprises an opening 104 that aligns with the second end of the tubular structure 101 to provide access to the interior of the tubular structure 101.

The opening 104 provides access to a mold cavity 106 of the hub 103 for holding a cap set therein. The mold cavity 106 includes an outer diameter and an inner diameter, wherein the outer diameter is greater than the inner diameter. The mold cavity 106 is shaped and dimensioned to fit a cap set therein. The hub 103 comprises a pair of elongated tabs 107 thereon, wherein the pair of elongated tabs 107 comprises a first tab and a second tab.

In the illustrated embodiment, the tabs 107 comprise rounded terminal ends. The tabs 107 extend outward in opposite directions so that they are substantially collinear to one another. The tabs 107 can be pulled apart such that the first tab and the second tab become separated. Accordingly, the hub 103 can comprise a seam where the first tab and the second tab meet and separate. In some embodiments, the tabs comprise ridges 102 thereon. The hub 103 further comprises a plurality of posts 105 that protrude around the opening 104 thereof, wherein the posts 105 are separated at regular intervals.

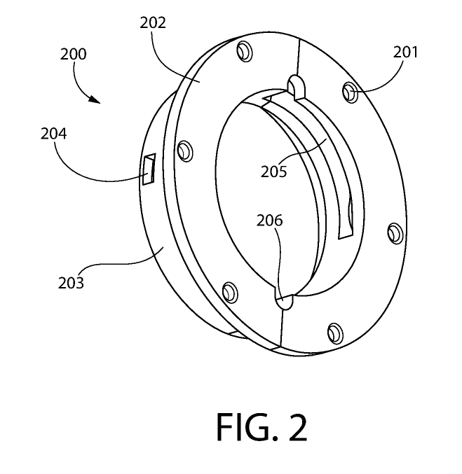

Referring now to FIG. 2, there is shown an exemplary embodiment of the cap set 200 of the present invention. The cap set 200 comprises a first section 203 and a second section 206 that are assembled together, wherein the first section 203 and the second section 206 are substantial mirror images of each other. In this regard, the cap set 200 comprises a seam where the first section 203 and the second section 206 meet and separate. Each of the sections 203, 206 comprises a semicircle so as to form an open circular shape or an annular shape when assembled together and viewed from the top. The opening in the center of the cap set 200 form a valve housing for retaining the valve therein.

In the illustrated embodiment, each of the sections 203, 206 comprises a semicylindrical section 204 having a flange 202 orthogonal thereto, wherein the flange 202 comprises a plurality of apertures 201 thereon. The outer perimeter of the flange 202 is greater than the outer perimeter of the semicylindrical section 204, while the inner perimeter of the flange 202 is equal to the inner perimeter of the semicylindrical section 204. The flange 202 is configured to fit within the outer diameter of the mold cavity 106 (FIG. 1) of the sheath hub 103 (FIG. 1) and the semicylindrical section 204 is configured to fit within the inner perimeter of the mold cavity 106 (FIG. 1) of the sheath hub 103 (FIG. 1).

It is noted that the two sections 203, 206 of the cap set 200 are not bonded or adhered together. Instead, the cap sections 203, 206 are held together when the semicylindrical section 204 of the cap sections 203, 206 are set into the mold cavity 106 (FIG. 1) at the opening 104 (FIG. 1) of the sheath hub 103 (FIG. 1) and the apertures 201 receive the posts 105 (FIG. 1) therethrough. In this regard, the apertures 201 are positioned so that each one aligns with one of the posts 105 (FIG. 1) disposed on the sheath hub 103 (FIG. 1) near the opening 104 (FIG. 1) thereof and receive the posts 105 (FIG. 1) at least partially therethrough, thereby securing the cap set 200 to the sheath hub 103 (FIG. 1) and preventing the cap set 200 from moving within the sheath hub 103 (FIG. 1).

Additionally, the semicylindrical section 204 of each of the sections 203, 206 can comprise one or more openings 205 or cutouts thereon for aligning within or fitting into the mold cavity 106 (FIG. 1) of the sheath hub 103 (FIG. 1). Similarly, the flange 202 can comprise one or more openings and/or cutouts that can align with other components of the present tearaway sheath introducer and/or a dilator.

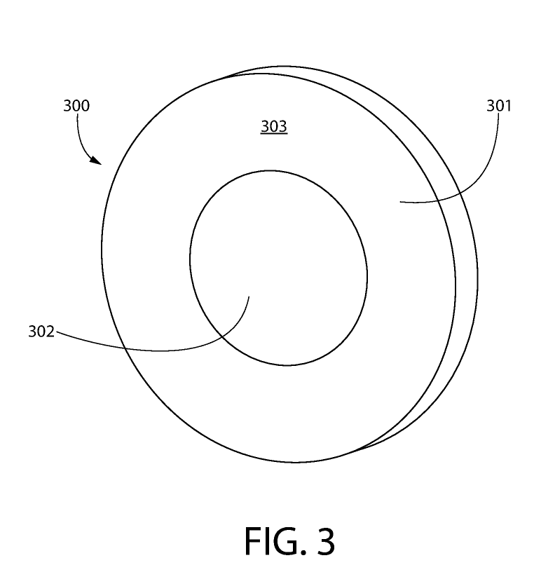

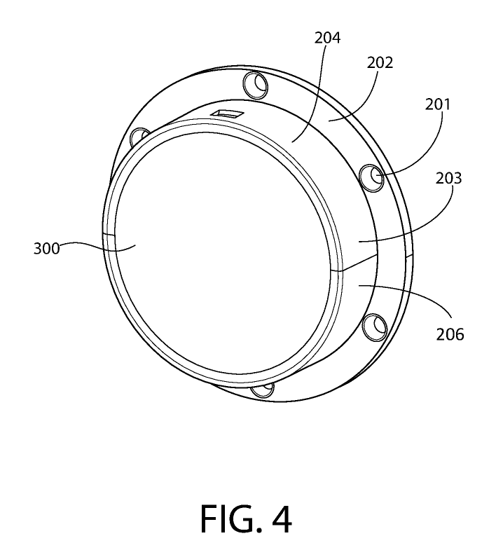

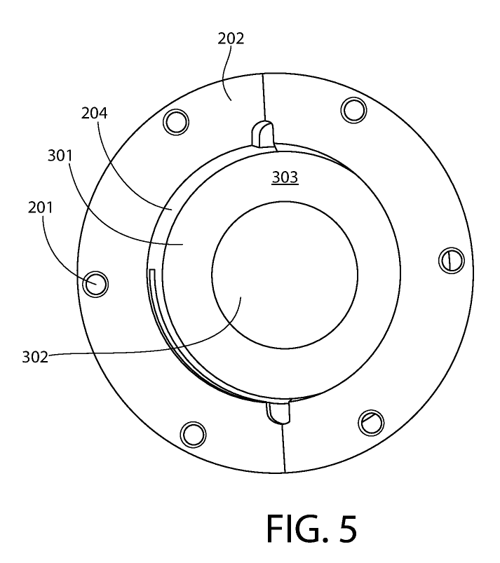

Referring now to FIG. 3, there is shown a view of the valve of the present invention. The valve 300 is composed of a resilient material that can be manipulated and returned to its normal position automatically. The valve 300 comprises a disk-like shape (e.g., having a circular cross section) with a defined front side 301 opposite a back side. The surface 303 of the front side 301 comprises a depressed portion 302 for allowing the dilator or a catheter to be inserted and exited therethrough. As shown in FIGS. 4 and 5, the valve material is injected into the mold cavity of the cap set 200 (FIG. 2) so as to hold the two cap sections 203, 206 together. In this way, the diameter of the valve 300 is substantially equal to the inner diameter of the semicylindrical section 204 and the flange 202 of the cap sections 200 (FIG. 2).

The back side of the valve 300 is biased toward the semicylindrical section 204 of the cap set 200 (FIG. 2) while the front side 301 of the valve 300 is biased toward the flange 202 of the cap set 200 (FIG. 1). In this way, the flange 202 surrounds the depressed portion 302 on the front surface 303 of the valve 300. The back side of the valve 300 is substantially coplanar or flush with the lower end of the semicylindrical section 204 of the cap set 200 (FIG. 2). The front side 301 of the valve 300 is slightly recessed into the valve housing of the cap set 200 (FIG. 2). The valve 300 can only separate when it is slitted across, preferably where the two cap sections 203, 206 meet and separate (i.e., the seam between the two sections 203, 206). It is noted that the valve 300 does not extend beyond the cap sections 203, 206. Additionally, the valve 300 is not molded separately. The cap set 200 (FIG. 2) and the valve 300 are placed into the sheath hub 103 (FIG. 1) with the posts 105 (FIG. 1) aligned with the apertures 201 (FIG. 2) disposed on the flanges 202 of the cap sections 203, 206, and then ultrasonically welded together.

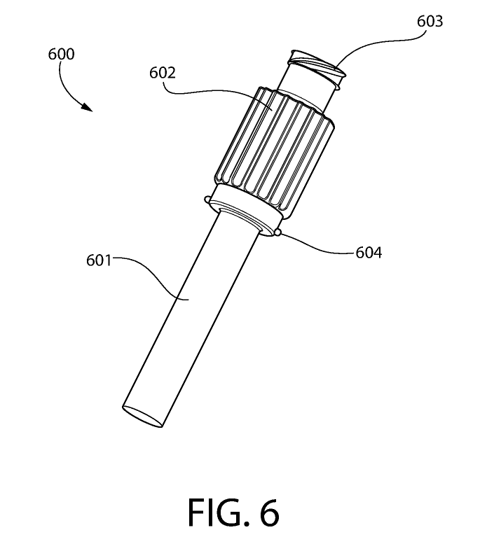

Referring now to FIG. 6, there is shown a view of the dilator 600 with a hub 602 that is configured to fit onto the cap set 200 (FIG. 2) and lock in place during insertion. The dilator 600 comprises a substantially tubular structure 601 with a circular cross-section, wherein the diameter of the dilator is slightly less than the diameter of the sheath 101 (FIG. 1) so as to allow the tubular structure 601 of the dilator 600 to be inserted into the tubular structure 101 (FIG. 1) of the sheath 100 (FIG. 1). Additionally, the dilator comprises a first end and a second end, wherein the second end comprises a hub 602 thereon.

The lower end 604 of the dilator hub 602 is configured to mate with the inner perimeter of the flange 202 (FIG. 2) of the cap set 200 (FIG. 2). In this regard, the lower end 604 of the dilator hub 602 comprises protrusions and/or cutouts that can be aligned with the protrusions and/or cutouts disposed on the flange 202 (FIG. 2) of the cap set 200 (FIG. 2). Furthermore, the hub 602 comprises a locking mechanism 603 (e.g., threaded elements) such that the dilator 600 can be temporarily locked or fitted to any apparatus that may be required to be attached to the dilator 600 during the insertion process.

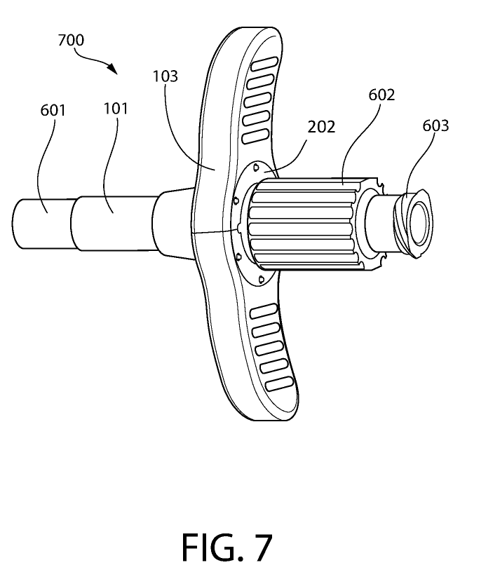

Referring now to FIG. 7, there is shown a view of the complete tearaway sheath introducer assembly 700. The tubular structure 601 of the dilator is inserted into the tubular structure 101 of the sheath so that the dilator hub 602 sits on the sheath hub 103. The distance between the first and second end of the tubular structure 601 of the dilator is greater than the distance between the first and second end of the tubular structure 101 of the sheath so that the first end of the tubular structure 601 of the dilator extends beyond the first end of the tubular structure 101 of the sheath.

The cap set and the valve are inserted into the mold cavity of the sheath hub 103. Thus, the tubular structure 601 of the dilator is inserted through the valve so as to allow the lower end of the dilator hub 602 to be positioned directly within the flange 202 of the cap set. In this regard, the lower end of the dilator hub 602 is adjacent to the valve. Additionally, because the valve is slightly recessed into the valve housing of the cap set, the lower end of the dilator hub 602 can be substantially coplanar or flush with the flange 202 of the cap set.

In use, the complete assembly 700 is inserted over a guide wire. The dilator can be connected to any apparatus via the locking mechanism 603 at a terminal end thereof. The dilator 601 is then removed and exited through the valve, leaving the sheath 101 in the blood vessel. Due to the resilient nature of the valve, the valve is configured to prevent blood from coming out after the dilator is removed, but before a catheter is inserted. Once the catheter is fully inserted through the valve and into the sheath, the sheath hub 103 is separated or broken into two halves at the seam, separating the two tabs.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A tearaway sheath introducer, comprising:
a sheath having a first open tubular structure comprising a first end and a second end, said second end of said first tubular structure comprising a sheath hub;
a dilator comprising a second open tubular structure comprising a first end and a second end, said second end of said second tubular structure comprising a dilator hub;
said first tubular structure configured to receive at least a portion of said second tubular structure therethrough;
a cap set comprising a valve molded thereon, wherein said cap set is recessed into said sheath hub, further wherein said valve is configured to prevent blood from coming out of a patient's incision site after said dilator is removed, but before a catheter is inserted.

2. The tearaway sheath introducer of claim 1, wherein said dilator hub comprises a locking mechanism at an upper end thereof.

3. The tearaway sheath introducer of claim 1, wherein said cap set and said valve are ultrasonically welded to said sheath hub.

4. The tearaway sheath introducer of claim 1, wherein said cap set comprises a first section and a second section, each of said first section and said second section comprising a substantially semicircular shape to form an annular shape with a valve housing when assembled together.

5. The tearaway sheath introducer of claim 1, wherein said sheath hub comprises a pair of tabs extending outward therefrom, wherein said pair of tabs can be separated.

6. The tearaway sheath introducer of claim 1, wherein said sheath hub comprises a central opening that is aligned with said second end of said sheath;
said central opening comprising a mold cavity for receiving said cap set therein.

7. The tearaway sheath introducer of claim 1, wherein said sheath hub comprises a central opening having a plurality of posts separated at regular intervals;
wherein said cap set comprises a plurality of apertures around a perimeter thereof;
said plurality of posts aligned with said plurality of apertures to secure said cap set onto said sheath hub.

8. The tearaway sheath introducer of claim 1, wherein said dilator hub is configured is positioned directly adjacent to said cap set.

9. A tearaway sheath introducer, comprising:
a sheath having a sheath hub attached thereto, wherein said sheath hub comprises an opening for providing access to a mold cavity;
said opening comprising a plurality of posts;
a cap set that is configured to fit within said mold cavity of said sheath hub, wherein said cap set comprises a first section and a second section that are assembled together to form a central opening that defines a valve housing to retain a valve molded therein;
each of said first section and said second section comprising a flange having a plurality of apertures aligned with said plurality of posts for receiving said plurality of posts therethrough when said cap set is positioned within said mold cavity of said sheath hub;
a dilator comprising a dilator hub attached thereto, wherein a lower end of said dilator hub is at least partially recessed into said cap set and positioned adjacent to said valve.

10. The tearaway sheath introducer of claim 9, wherein said sheath comprises a first tubular structure having a first end and a second end, said second end of said sheath comprising said sheath hub.

11. The tearaway sheath introducer of claim 10, wherein said dilator comprises a second tubular structure having a first end and a second end, said second end of said second tubular comprising said dilator hub.

12. The tearaway sheath introducer of claim 11, further wherein said second tubular structure is configured to fit through said first tubular structure.

11. The tearaway sheath introducer of claim 9, wherein said dilator hub comprises a locking mechanism.

12. The tearaway sheath introducer of claim 9, wherein said cap set and said valve are ultrasonically welded to said sheath hub.

13. The tearaway sheath introducer of claim 9, wherein said sheath hub comprises a pair of tabs extending outward therefrom.

14. The tearaway sheath introducer of claim 13, wherein said pair of tabs comprise a first tab and a second tab that can separate at a seam therebetween when pulled apart.

15. A tearaway sheath introducer, comprising:
a sheath having a first open tubular structure comprising a first end and a second end, said second end of said first tubular structure comprising a sheath hub, wherein said sheath hub comprises an opening for providing access to a mold cavity;
said opening comprising a plurality of posts therearound;
a cap set that is configured to fit within said mold cavity of said sheath hub, wherein said cap set comprises a first section and a second section that are assembled together to form a central opening that defines a valve housing to retain a valve molded therein;
each of said first section and said second section comprising a flange having a plurality of apertures aligned with said plurality of posts for receiving said plurality of posts therethrough when said cap set is positioned within said mold cavity of said sheath hub;
a dilator comprising a second open tubular structure comprising a first end and a second end, said second end of said second tubular structure comprising a dilator hub, wherein a lower end of said dilator hub is at least partially recessed into said cap set and positioned adjacent to said valve when said second open tubular structure is inserted through said first open tubular structure.

16. The tearaway sheath introducer of claim 15, wherein said mold cavity includes an outer diameter and an inner diameter;
said outer perimeter greater than said inner diameter.

17. The tearaway sheath introducer of claim 16, wherein each of said first section and said second section comprises a semicylindrical section that is orthogonal to said flange;
said semicylindrical section configured to fit within said inner diameter;
said flange configured to fit within said outer diameter.

18. The tearaway sheath introducer of claim 15, wherein said second open tubular structure extends beyond said first open tubular structure.

19. The tearaway sheath introducer of claim 15, wherein said valve is recessed within said cap set so as to allow said lower end of said dilator hub to sit flush with said flange.

20. The tearaway sheath introducer of claim 15, wherein said sheath hub comprises a pair of tabs extending outward therefrom;
said pair of tabs comprising a first tab and a second tab that can separate at a seam therebetween when pulled apart.
